(19) United States
(12) Patent Application Publication
(10) Pub. No.: US 2020/0380462 A1
(43) Pub. Date: Dec. 3, 2020

(54) Method for monitoring bituminous material

(71) Applicant: Jeremy Alan Fenolio, Crown Point, IN (US)

(72) Inventor: Jeremy Alan Fenolio, Crown Point, IN (US)

(21) Appl. No.: 16/426,510

(22) Filed: May 30, 2019

(51) Int. Cl. G06Q 10/08; G06Q 50/30; G01N 33/42; C08L 95/00

(52) U.S. Cl. CPC G06Q 10/0833; G06Q 50/30; C08L 2555/10; C08L 95/00; G01N 33/42

(57) ABSTRACT

A method by computer useful for monitoring the environmental characteristics of bituminous material that is used in an industrial process. Disclosed is a particularly useful method for the processing of asphalt when a transport takes the bituminous material under its control. The method being capable of communicating environmental information related to the material as observed by the transport and the users involved in the industrial process.

### CROSS-REFERENCE TO RELATED APPLICATIONS

The present application claims the benefit of U.S. Provisional Patent Application No. 62/679,220, filed Jun. 1, 2018, the entirety of which is hereby incorporated by reference.

### BACKGROUND

Every day, industrial companies produce goods and deliver these goods to their customers. Delivering their goods mostly involves various means of ground transportation. The companies employ truck drivers to transport goods from one location to another. In the construction industry, dump trucks are used for hauling material to job sites and back again to the main plant. This is especially true of plants that process bituminous material such as asphalt plants.

Hauling causes drivers and their trucks to be away from their employer's main offices. This places these resources outside of the control of plant management and provides very little information to management about the bituminous material or activities the drivers and their trucks are doing while on the road. Importantly, constant monitoring of the state of the bituminous material throughout the plant process is done today manually, or not at all.

Certain technologies allow company mangers to get information about their trucks and drivers while the trucks are away from the plant. Telephones, cameras, computers, CB radios, and geosynchronous positioning satellite devices can provide regular updates to management regarding the whereabouts of their trucks. However, these devices are designed to collect regular and predictable data. These cannot handle the varied information, often unpredictable, related to a truck and its driver, and importantly the bituminous material being processed. Additionally, these technologies do not integrate easily with the processes, procedures, and the data systems used by other resources in the company. Instead, the company's systems must adapt to the technology used.

Information relevant to attempts to address these problems can be found in public articles such as that presented at https://www.hcss.com/products/construction-truck-ticket-management/ (last visited May 28, 2019). However, each of the references cited therein, that were available for review, suffer from one or more of the following disadvantages: 1) they do not provide a process for a transport and computer together constantly monitoring the state of bituminous material, 2) the software is not collecting all possible characteristics of the material and transport, and 3) such solutions focus on the creation of tickets.

For the foregoing reasons, there is a need in the industry for a solution to constantly monitor the state of bituminous material, to more thoroughly link a transport, such as a dump truck and its driver, to the policies, procedures, people and data processing systems that are part and parcel of an industrial company's business. One that is able to communicate information about truck and driver interactions whether predictable or not. A method to allow complete communication of information related to the handling of bituminous material amongst all users involved in an industrial plant process.

Additionally the needed process would make the details of tracking bituminous material in an industrial process more accurate for those involved and allow for questions on the state of that material to be asked and answered. The needed process would allow for accurately pinpointing exact quantities of material and would assist with answering questions such as “where is the material?”, “who has the material?”, “has the material been placed at a locality?”, “how much?” or “is the material still hot?” to name a few examples.

### SUMMARY

The present invention is directed to a computer-based solution that satisfies these needs. The solution comprises a transport, such as a company's truck and driver, equipped with a software driven device, a computer. The software driven device is specially adapted to allow the driver to interact freely with the device. The device communicates these interactions to a company's normal business systems and processes that are used at the company's main office.

The solution is used in asphalt company processes that require management control over truck and driver interactions that are often unpredictable to name one example.

I have invented a method by computer for monitoring the movement of bituminous material that is used in an industrial plant process operated by many disparate users. I envision the method being best used in an asphalt plant.

One version of the inventive method comprises programming the computer. A programmer, or group of programmers and data entry personnel, working in the industrial plant, will populate the computer, which need not be a single computer but could be a network of computers, with computer logic and data in a manner so as to fully inform the computer as to all steps needed to complete the industrial plant process. Once programmed, the computer will have an artificial intelligence to allow it to make logic computations, decisions, using data related to the process. The data fields of the program would include pieces of information that are specific to the processing of bituminous material.

A next step in the method involves connecting a transport to the computer by placing the transport substantially near the computer in a manner so as to allow authentication and two way communication. The transport can be an automated machine such as a self driving and intelligent vehicle.

I envision a best mode of the invention for this step comprising a transport that is a human being, such as a commercially licensed truck driver that is a union member, along with an assigned dump truck. The connection is best accomplished via a screen login and authentication process between the transport and the computer. Authentication could be accomplished without human interaction, such as via an automated machine transport making connection via an electronic handshake with the computer.

Via the connection, in one embodiment of the inventive computer-based method, the computer is able to combine information, combining a union driver's and a dump truck's identifying information with the bituminous material that is later associated with that transport for example, and display the combined information on an electronic hardware device as needed.

Next, the computer will deliver to the transport at least one first datum that relates to a first quantity of bituminous material to be transported. The bituminous material is best if it is asphalt, however, it can be aggregate, tar, stone, sand, concrete, or any such material used in construction. The at least one first datum is delivered to the transport in any of a multitude of means. I envision the best delivery method to be via an electronic display. Though, if the transport included an automated machine instead of a human driver then such a display is not be needed.

The at least one first datum can include information about the first quantity of bituminous material to be transported that is commonly understood by users in an industrial plant, such as an asphalt plant for example. I envision a large portion of this data being retrieved from software that is integrated into the industrial plant process. For instance there may be separate software that is used to create “tickets” related to the bituminous material. Traditionally, these tickets are handed to a driver who then acts on the information printed on the ticket.

Such information delivered might be quantity, type, weight, where the material is located, temperature, particle size, brand name, and color to name a few examples. Knowing information about the bituminous material's environment is important to carrying out the industrial process.

Then the computer will request that the transport take control of the first quantity of bituminous material to be transported. Taking control often involves a transport entering into a receiving bin where material has been mixed, stored, and readied for placement into a transport's receiver, the dump on a dump truck for instance. Too, the material can be placed into the transport's receiver by another machine, such as a front end loader, which often happens for digouts.

The computer then awaits a first confirmation from the transport that the first quantity of bituminous material to be transported is under the transport's control. It is important that this confirmation be completed so as to communicate to the computer that the material is about to change its environmental characteristics, e.g. the location will soon change. This confirmation can be completed automatically, using wireless communicating servo-mechanisms for instance, in the case of an automated machine transport. I envision the confirmation can be completed via human interaction with the computer in the case of a transport comprising a human driver.

Once the first quantity of bituminous material to be transported is under the control of the transport, it is important to advise the transport of new information identifying that material. So a next step is for the computer to deliver to the transport at least one second datum that relates to, at least, a locality where the first quantity of bituminous material to be transported is to be processed. The data transferred can include more information about the material as well.

Then the transport's function in the industrial plant process is to deliver the first quantity of bituminous material to be transported and have it placed at the locality, for example a job site specific to a customer of the industrial plant. Examples, of such job sites are envisioned to be a road to be paved, a building, or some other similar type of construction project that requires the use of asphalt.

Once placed at the locality, it important to have the computer electronically record the information that pertains to the first quantity of bituminous material to be transported both before and after it was placed. The next step is electronically storing the at least one first datum, the at least one second datum, and a third data set that corresponds to the first confirmation and the second confirmation.

The inventive method described above uses a computer to continuously monitor the first quantity of bituminous material to be transported as it is processed by an industrial plant. The method can be repeated for other quantities of bituminous material to be transported and also duplicated for other computer/transport pairs. Due to the constant monitoring, an embodiment of the invention can combine information encountered by a driver and thereafter entered into a computer via an electronic hardware device or combine information collected by an automatic measuring device. It is envisioned that an app can be used by the driver to as the electronic hardware device, there is nothing novel about this approach for entering data into the computer, however, this method of continuously monitoring the bituminous material in this manner is not known.

Another embodiment of the inventive method, at the step of awaiting a first confirmation can further comprise the computer monitoring a plurality of environmental indicators that relate to the state of the first quantity of bituminous material to be transported. Such environmental indicators include, without limitation, the weight, amount, temperature, location, thickness, water content, and the like to name a few examples. These indicators are communicated to the computer via automatic means, such as via electronic measuring devices that are commonly used, but I envision this being communicated by the transport after the transport's observations of changing environmental indicators. “I accidentally lost the load of material and it is dispersed all over the field” might be information communicated by a transport.

The process would go on to alert the disparate users upon the occurrence of any of the plurality of environmental indicators substantially causing the industrial plant process to become at risk of non-completion. Users in the industrial plant process are adept at understanding what will and will not stall a process. A driver, in a human transport, is not only part of the transport but also one of the disparate users involved in the process.

The computer, though fully informed in the best mode as envisioned by me, still requires a user's decision making skills to be communicated to the computer. So the method allows for a step where the computer can capture a response from the disparate users related to the alerting and based upon the response from the disparate users, act in some way on the material, remove the first quantity of bituminous material to be transported from the transport's control to name one common example. An example of when this might happen is if a transport becomes inoperative after taking control of the bituminous material. Certainly, other decisions can be made depending on the effect the alerting will have on the completion of the industrial process.

I envision another embodiment, where the step of electronically storing further comprises electronically processing the data electronically stored. This processing is done by the program logic of the computer. Then presenting detailed information that corresponds to the processed data to the disparate users in a manner so as to allow the users to determine whether an oral communication with another user or with the transport should be initiated. The same idea applies to allow the transport to determine whether an oral communication with another user should be initiated, a two-way transport-to-dispatcher communication for example.

Yet another embodiment of the inventive method involves using a computer for monitoring the movement of asphalt that is used in an asphalt plant process operated by many disparate users, the method comprising programming the computer with data in a manner so as to fully inform the computer as to all steps needed to complete the asphalt plant process. Then connecting a union member truck driver, ideally a Teamster member, operating a dump truck to the computer by placing the union member truck driver and dump truck substantially near the computer in a manner so as to allow authentication and two way communication.

Next is delivering from the computer to the union member truck driver and dump truck at least one first datum that relates to a first quantity of asphalt to be transported. Then requesting that the union member truck driver and dump truck take control of the first quantity of asphalt to be transported. Taking such control allows the driver and truck to be in constant observance of the environmental characteristics of the asphalt.

Next is awaiting a first confirmation from the union member truck driver and dump truck that the first quantity of asphalt to be transported is under the union member truck driver and dump truck's control. Then delivering to the union member truck driver and dump truck at least one second datum that relates to a locality where the first quantity of asphalt to be transported is to be processed. This would likely include the job site where the asphalt is to be laid.

Then awaiting a second confirmation from the union member truck driver and dump truck that the first quantity of asphalt to be transported has been placed at the locality. Next is electronically storing the at least one first datum, the at least one second datum, and a third data set that corresponds to the first confirmation and the second confirmation. The computer then allows for monitoring a plurality of environmental indicators that relate to the state of the first quantity of asphalt to be transported. Next step in the method is alerting the disparate users, one or many, upon the occurrence of any of the plurality of environmental indicators substantially causing the industrial plant process to become at risk of non-completion. A digout or a spill or a breakdown for instance. Then capturing a response from the disparate users related to the alerting and based upon the response from the disparate users, removing the first quantity of asphalt to be transported from the union member truck driver and dump truck's control. Electronically processing the data electronically stored and then presenting detailed information that corresponds to the processed data to the disparate users in a manner so as to allow the users to determine whether an oral communication with another user or with the union member truck driver and dump truck should be initiated.

Too the next step could involve presenting detailed information that corresponds to the processed data to the union member truck driver and dump truck in a manner so as to allow the union member truck driver and dump truck to determine whether an oral communication with another user should be initiated. The method allows obtaining a substantial portion of the at least one first datum from a software system selected from the group consisting of JWS, Astec, and Viewpoint.

Several examples of objectives for the inventive method are: to allow for manual entry of job information, such as information characteristic of a digout or dumpsite to name a few examples, into an electronic hardware device. To allow for tracking heavy equipment data related to a job, such as loads on a digout to name one example. To be able to collect information from a vehicle driver or a recording computer related to incidents that are not ordinary, break downs or delays to name two examples. To allow a driver to quickly assign a value to a yes or no question via interaction with an electronic hardware device. For instance, did you take a lunch? Answer yes or no. To convert a paper based process into a process with less paper product delivered between material collection points thereby minimizing data entry, streamlining payroll timecards of dump truck drivers, and entering job cost data to name just two examples. To be able to bring together electronic data from different sources. To simplify electronic data input creating near real time data analysis and providing increased data accuracy. To allow remote logging of activities, logging vehicle drivers as being “in” or “out” to name one example. To assist users with rule compliance, such as assisting with ELD mandate if a transport travels outside a one hundred mile radius to name one example. Lastly, another example of an objective of the inventive method is to incorporate a dump truck into a company's data processing system such that a myriad of attributes related to the dump truck are communicated to the data processing system via an electronic hardware device.

### BRIEF DESCRIPTION OF THE SEVERAL VIEWS OF THE DRAWINGS

These and other features, aspects and advantages of the present invention will become better understood with regard to the following description, appended claims, and accompanying drawings where:

FIG. 1 provides a block diagram of one embodiment of the present invention.

FIG. 2. depicts a flow chart of another embodiment of the present invention.

FIG. 3 provides a block diagram of another embodiment of the present invention.

### DESCRIPTION

### Overview.

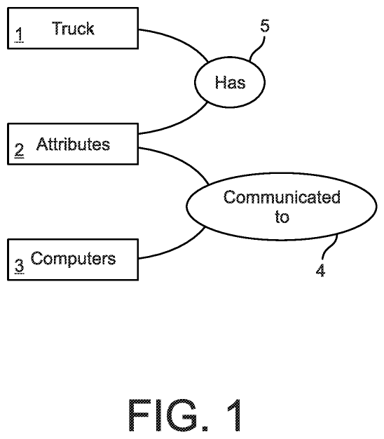

As shown in FIG. 1, a block diagram of one embodiment of the invention comprises a transport 1, such as a dump truck being driven by a union truck driver to name one example of a transport. The transport 1 is of a nature described 5 by many attributes 2. Such attributes, sometimes herein referred to as environmental characteristics, describe not only the physical attributes of the transport itself but also environmental characteristics of a bituminous material that is under the control of the transport. As the transport 1 works as directed to do so by the industrial plant process, there are a multitude of physical attributes 2 that describe the nature of the transport 1 at any given moment in time. These attributes 2 are communicated to 4 one or many data processing machines or computers 3. The attributes 2 can also be communicated to 4; a company's other business resources, such as the users involved in the industrial process, those involved in asphalt processing for instance.

Attributes 2 common to an asphalt processing process are the type of material, its weight, the union driver operating the dump truck, identifying information for the dump truck, the electronic connection attributes of the transport, the temperature, location, and the like to name a few examples of attributes, or environmental characteristics, common to the process.

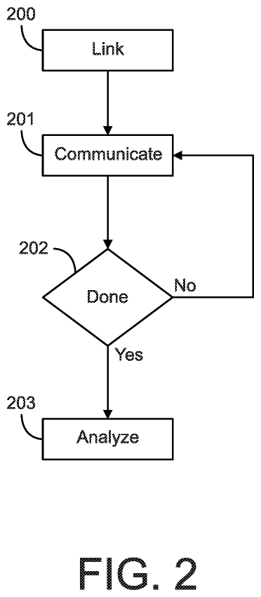

As shown in FIG. 2, a block diagram of another embodiment of the invention comprises making a link of entities 200. It is envisioned that the best mode is to link a dump truck, a dump truck driver, knowledge of an industrial company's policies and procedures, and a data processing system. The dump truck driver will communicate 201 observed characteristics related to these linked 200 entities to a computer system. It is envisioned that the best mode for doing this communication 201 is via a human controllable electronic device, a computer with a touchscreen to name one example. A driver will interact with the linked 200 entities throughout a day making decisions as to whether the communications are completed or not 202. When there is no longer a need to communicate the information communicated 201 can be analyzed electronically 203 by a data processing system.

This embodiment of the inventive method uses a computer to monitor the movement of bituminous material that is used in an asphalt plant process and is operated by many disparate users. The computer is programmed with information on all steps needed to complete the asphalt plant process. Then the link 200, or connection, couples a transport, the union driver and dump truck, to the computer by placing the transport substantially near the computer in a manner so as to allow authentication and two way communication 201. Once there is no longer a need for such communications the user and the computer will analyze 203 the data corresponding to the communications.

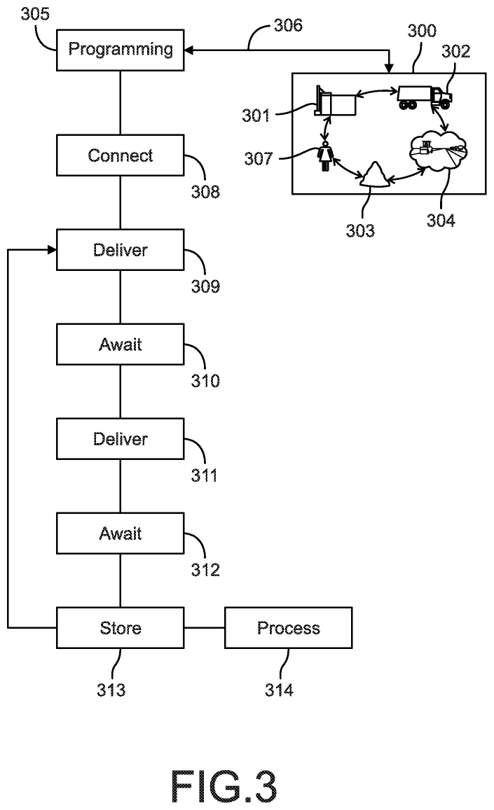

As shown in FIG. 3, a block diagram of another embodiment of the present invention comprises a method by computer for monitoring the movement of bituminous material that is used in an industrial plant process 300 operated by many disparate users 307. The users 300 will program 305 the computer with data in a manner so as to fully inform the computer as to all steps needed to complete the industrial plant process 300.

A connection 308 is made between a transport, a union truck driver operating a dump truck for example, to the computer by placing the transport substantially near the computer in a manner so as to allow authentication and two way communication. There is a first delivery 309 from the computer to the transport of at least one first datum that relates to a first quantity of bituminous material to be transported. Then the computer or users can request that the transport take control of a first quantity of asphalt to be transported and await 310 a first confirmation from the transport that the first quantity of asphalt to be transported is under the transport's control.

Then there is a second delivery 311 of data to the transport of at least one second datum that relates to a locality where the first quantity of asphalt to be transported is to be processed. The users or compute await 312 a second confirmation from the transport that the first quantity of asphalt to be transported has been placed at the locality.

The computer will electronically store 313 the data communicated and make the data available for processing by the asphalt processing.

Although the present invention has been described in considerable detail with the reference to certain preferred versions thereof, other versions are possible. For example, the transport can be a self driving vehicle without a human driver, too the method can be performed in any sequence of the steps disclosed. Therefore, the spirit and scope of the appended claims should not be limited to the description of the preferred versions contained herein.

Any element in a claim that does not explicitly state “means for” performing a specified function, or “step for” performing a specific function, is not to be interpreted as a “means” or “step” clause as specified in 35 U.S.C. § 112, ¶6. In particular, the use of “step of” in the claims herein is not intended to invoke the provisions of 35 U.S.C. § 112, ¶6.

What I claim is:

1. A method by computer for monitoring the movement of bituminous material that is used in an industrial plant process operated by many disparate users, the method comprising:
programming the computer with data in a manner so as to fully inform the computer as to all steps needed to complete the industrial plant process;
connecting a transport to the computer by placing the transport substantially near the computer in a manner so as to allow authentication and two way communication;
delivering from the computer to the transport at least one first datum that relates to a first quantity of bituminous material to be transported;
requesting that the transport take control of the first quantity of bituminous material to be transported;
awaiting a first confirmation from the transport that the first quantity of bituminous material to be transported is under the transport's control;
delivering to the transport at least one second datum that relates to a locality where the first quantity of bituminous material to be transported is to be processed;
awaiting a second confirmation from the transport that the first quantity of bituminous material to be transported has been placed at the locality;
electronically storing the at least one first datum, the at least one second datum, and a third data set that corresponds to the first confirmation and the second confirmation.

2. The method of claim 1 wherein the step of awaiting a first confirmation further comprises:
the computer monitoring a plurality of environmental indicators that relate to the state of the first quantity of bituminous material to be transported;
alerting the disparate users upon the occurrence of any of the plurality of environmental indicators substantially causing the industrial plant process to become at risk of non-completion;
capturing a response from the disparate users related to the alerting.

3. The method of claim 2 wherein based upon the response from the disparate users, removing the first quantity of bituminous material to be transported from the transport's control.

4. The method of claim 1 wherein the step of electronically storing further comprises:
electronically processing the data electronically stored;
presenting detailed information that corresponds to the processed data to the disparate users in a manner so as to allow the users to determine whether an oral communication with another user or with the transport should be initiated.

5. The method of claim 1 wherein the step of electronically storing further comprises:
electronically processing the data electronically stored;
presenting detailed information that corresponds to the processed data to the transport in a manner so as to allow the transport to determine whether an oral communication with another user should be initiated.

6. The method of claim 1 further comprising processing asphalt according to the industrial plant process the asphalt being useful for application on roadways.

7. The method of claim 1 wherein the step of delivering from the computer to the transport at least one first datum further comprises:
obtaining a substantial portion of the at least one first datum from a 3rd party software system that is incorporated into the industrial plant process.

8. A method by computer for monitoring the movement of asphalt that is used in an asphalt plant process operated by many disparate users, the method comprising:
programming the computer with data in a manner so as to fully inform the computer as to all steps needed to complete the asphalt plant process;
connecting a union member truck driver operating a dump truck to the computer by placing the union member truck driver and dump truck substantially near the computer in a manner so as to allow authentication and two way communication;
delivering from the computer to the union member truck driver and dump truck at least one first datum that relates to a first quantity of asphalt to be transported;
requesting that the union member truck driver and dump truck take control of the first quantity of asphalt to be transported;
awaiting a first confirmation from the union member truck driver and dump truck that the first quantity of asphalt to be transported is under the union member truck driver and dump truck's control;
delivering to the union member truck driver and dump truck at least one second datum that relates to a locality where the first quantity of asphalt to be transported is to be processed;
awaiting a second confirmation from the union member truck driver and dump truck that the first quantity of asphalt to be transported has been placed at the locality;
electronically storing the at least one first datum, the at least one second datum, and a third data set that corresponds to the first confirmation and the second confirmation;
via the computer, monitoring a plurality of environmental indicators that relate to the state of the first quantity of asphalt to be transported;
alerting the disparate users upon the occurrence of any of the plurality of environmental indicators substantially causing the industrial plant process to become at risk of non-completion;
capturing a response from the disparate users related to the alerting;
based upon the response from the disparate users, removing the first quantity of asphalt to be transported from the union member truck driver and dump truck's control;
electronically processing the data electronically stored;
presenting detailed information that corresponds to the processed data to the disparate users in a manner so as to allow the users to determine whether an oral communication with another user or with the union member truck driver and dump truck should be initiated;
presenting detailed information that corresponds to the processed data to the union member truck driver and dump truck in a manner so as to allow the union member truck driver and dump truck to determine whether an oral communication with another user should be initiated;
obtaining a substantial portion of the at least one first datum from a software system selected from the group consisting of JWS, Astec, and Viewpoint.

9. The method of claim 8 wherein the union member truck driver is a Teamster.
